(19) United States
(12) Patent Application Publication
(10) Pub. No.: US 2024/0108499 A1
(43) Pub. Date: Apr. 4, 2024

(54) METHODS AND APPARATUS FOR APPLICATION OF FOAMED EYE DROPS

(71) Applicant: TWENTY TWENTY THERAPEUTICS LLC, South San Francisco, CA (US)

(72) Inventors: Oleg Rumyantsev, Menlo Park, CA (US); Dimitri Azar, Chicago, IL (US); Allister McGuire, South San Francisco, CA (US); Alexandre Tumlinson, South San Francisco, CA (US); Maxim Armstrong, South San Francisco, CA (US)

(21) Appl. No.: 18/367,825

(22) Filed: Sep. 13, 2023

Related U.S. Application Data
(60) Provisional application No. 63/406,034, filed on Sep. 13, 2022.

(51) Int. Cl. A61F 9/00

(52) U.S. Cl. CPC A61F 9/0008

(57) ABSTRACT

A device for delivering foamed eye medication to an eye includes a housing defining an interior space for receiving liquid eye medication and air. A nozzle has a passage in fluid communication with the interior space. A foaming chamber is positioned within the interior space and in fluid communication with the nozzle. The air and liquid eye medication are mixed in the foaming chamber and exit the nozzle as foamed medication in response to pressurizing the interior space.

### RELATED APPLICATIONS

This application claims the benefit of U.S. Provisional Application Ser. No. 63/406,034, filed Sep. 13, 2022, the entirety of which is incorporated by reference herein.

### TECHNICAL FIELD

The present invention relates generally to eye medication, and specifically to systems and methods for delivering foamed eye medication.

### BACKGROUND

The standard method for delivering medication to the eye of a patient is through the application of eye drops. A typical eye drop has a volume of, for example, about 25-75 microliters. The majority of this volume, however, can be wasted during drop application. A drop delivered to the eye forms puddles at the boundary between the sclera/cornea and the eyelid but the drug may not have enough time to diffuse through the cornea as the excess volume of the drop is removed when the person blinks. If the user does not look vertically up without blinking, the effectiveness of the drug may be significantly reduced.

### SUMMARY

The present invention relates to a new and improved ophthalmic device for delivering foamed medication to a user/patient's eye. The medication can be, for example, chemically foamed to enable foaming of the eye drop during application, i.e., the medication is already mixed with an optimal concentration of surfactants such that foaming is enhanced (as opposed to being suppressed) during application with any of the devices shown and described. The foamed medication can applied on the eye similar to an eye drop (gravitationally) or similar to an ointment (spread along the lower eyelid margin). A foam injector (similar to a syringe) may also be useful for delivering the medication horizontally. The volume, ratio of air to drug volumes, concentration of drugs, total drug weight/mass, and/or pH can all be modified to allow optimal residence time on the eye and reduce waste of medication.

The device can be configured to deliver a precise, repeatable foamed medication dosage in the range of about 100 to about 1000 μL. The dosage could consist of, for example, about 10-75 μL of liquid medication and the remainder gas. In this manner, the same volume of foamed medication can be delivered even as the available medication within the device is reduced with repeated usage.

In one example, a device for delivering foamed eye medication to an eye includes a housing defining an interior space for receiving liquid eye medication and air. A nozzle has a passage in fluid communication with the interior space. A foaming chamber is positioned within the interior space and in fluid communication with the nozzle. The air and liquid eye medication are mixed in the foaming chamber and exit the nozzle as foamed medication in response to pressurizing the interior space.

In another example, a device for delivering foamed eye medication to an eye includes a housing defining an interior space for receiving liquid eye medication and pressurized gas. An nozzle has a passage in fluid communication with the interior space. A foaming chamber is positioned within the interior space and in fluid communication with the nozzle. A valve is positioned between the foaming chamber and the nozzle. The pressurized gas and the medication mixes within the foaming chamber in response to actuation of the valve and exits the nozzle as foamed medication.

In another example, a device for delivering foamed eye medication to an eye includes a housing defining an interior space for receiving a pre-mixed mixture of liquid eye medication and a surfactant for chemically foaming the liquid eye medication. A nozzle includes a passage in fluid communication with the interior space for delivering foamed medication.

In another example, a device for delivering foamed eye medication to an eye includes a housing defining an interior space for receiving liquid eye medication. An adapter is connected to the housing and includes a first chamber for receiving air and a second chamber for receiving the medication. A nozzle includes a passage in fluid communication with the interior space. A foaming chamber is positioned within the adapter and in fluid communication with the nozzle. A pump draws the air into the first chamber while drawing the medication into the second chamber and subsequently delivering both the air and the medication to the foaming chamber such that the air and the medication mix and exit the nozzle as foamed medication.

Other objects and advantages and a fuller understanding of the invention will be had from the following detailed description and the accompanying drawings.

### BRIEF DESCRIPTION OF THE DRAWINGS

FIG. 1 is a schematic illustration of an example foamed eye medication dropper in accordance with the present invention.

FIG. 2 is a schematic illustration of another example foamed eye medication dropper.

FIG. 3 is a schematic illustration of another example foamed eye medication dropper.

FIG. 4 is a schematic illustration of an example foamed eye medication dropper.

FIG. 5 is a schematic illustration of an example foamed eye medication dropper.

### DETAILED DESCRIPTION

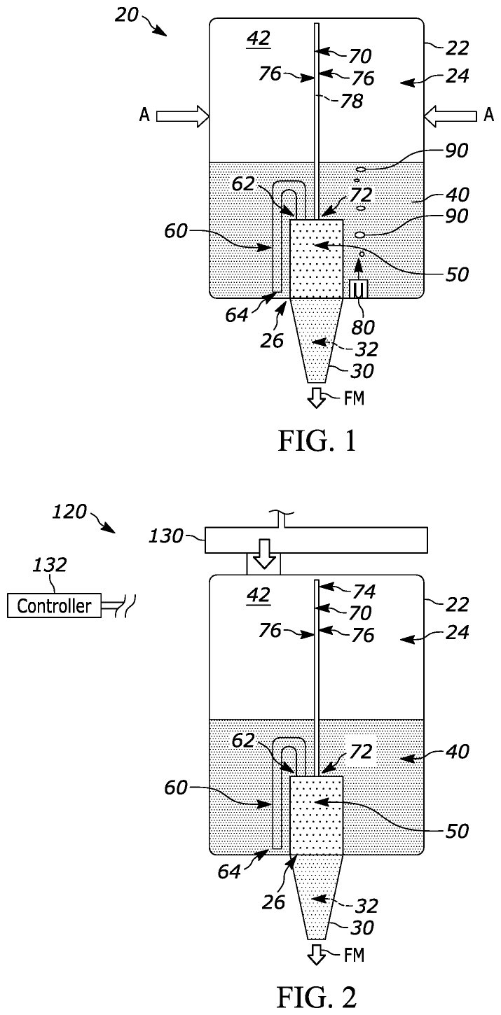

The present invention relates generally to eye medication, and specifically to systems and methods for delivering foamed eye medication. FIG. 1 illustrates an example ophthalmic device 20 in accordance with the present invention. The device 20 includes a housing 22 defining a chamber or interior space 24. An opening 26 extends through the housing 22 and into fluid communication with the chamber 24. A nozzle 30 is secured to or formed integrally with the housing 22. The nozzle 30 defines a passage 32 in fluid communication with the opening 26 and, thus, in fluid communication with the chamber 24.

Eye medication 40 is provided in the chamber 24. The medication 40 can constitute any liquid eye medication known to those having ordinary skill in the art, such as a therapeutic agent. The medication 40 only partially fills the chamber 24. The rest of the chamber 24 (above the medication 40 as shown) is filled with air 42.

A foaming chamber 50 is provided in the chamber 24 adjacent the opening 26. The foaming chamber 50 can be formed from a mesh lattice (e.g., a fine mesh lattice) and/or a porous foam material (e.g., a sponge material). A first or air intake tube 60 extends from a first end 62 positioned in the foaming chamber 50 to a second end 64 positioned within the medication 40. A second or drug intake tube 70 extends from a first end 72 positioned in the foaming chamber 50 to a second end 74 positioned within the air 42 in the chamber 24. Openings 76 are formed along the length of the tube 70 and extend to a common central passage 78.

A valve 80 is provided on the housing 22 and establishes one-way fluid communication from the exterior of the housing to the chamber 24. More specifically, the valve 80 allows air to pass from outside the device 20 and into the chamber 24, but prevents flow in the opposite direction. In one example, the valve 80 extends into the bottom (as shown) of the housing 22.

In one aspect of the present invention, the medication 40 is foamed by forcing it through the foaming chamber 50 while simultaneously introducing air 42 into the foaming chamber. When this occurs, the air 42 and medication 40 are mixed within the foaming chamber 50 and exit the foaming chamber as foamed medication FM. This, in turn, is expelled out of the nozzle 30 and into the user's/patient's eye.

With this in mind, in the example device 20 of FIG. 1, the user applies inward pressure to the housing 22, as indicated by the arrows A, which squeezes the housing. Consequently, the air 42 is compressed and urged into the openings 76 in the second tube 70, into the central passage 78, and ultimately into the foaming chamber 50 via the first end 72. At the same time, squeezing the housing 22 in the manner A urges the medication 40 into the second end 64 of the first tube 60, through the first tube, and ultimately into the foaming chamber 50 via the first end 62. The pressurized air 42 combines within the incoming medication 40 and exits the foaming chamber 50 and ultimately the nozzle 30 as a foamed medication FM. The volume of air 42 used to foam the medication 40 can be replenished through the valve 80 as the housing 22 expands/returns to its original, pre-squeezed condition. The replenishing air is indicated generally at 90.

Another example device 120 is shown in FIG. 2. In FIG. 2, features that are the same or substantially the same as those shown in FIG. 1 are given the same reference number. In FIG. 2, the pressurized air 42 is provided by a pump 130 operated by the user and fluidly connected to the chamber 24. In particular, the pump 130 pushes pressurized air 42 into the chamber 24 and, thus, into the openings 76 in the second tube 70. The pressurized air 42 also acts on the medication 40 by forcing it through the first tube 60 and into the foaming chamber 50. The pressurized air 42 combines with the incoming medication 40 and exits the foaming chamber 50 and ultimately the nozzle 30 as foamed medication FM. The pump 130 can cooperate with a controller 132 to drive a predetermined amount of air 42 into the foaming chamber 50 with each actuation.

It will be appreciated that the pump 130 could be replaced with a piston (not shown) having a defined stroke into the chamber 24 in order to pressurize the air 42 therein. In such a configuration, the controller 132 could actuate the piston to pressurize the air 42 and force the pressurized air and medication 40 into the foaming chamber 50. The piston can be configured to pressurize a defined volume of air 42 during each application (rather than a volume that changes as the volume of the medication 40 decreases over time). This would help ensure a uniform or substantially uniform volume of foamed medication 40 is delivered to the nozzle 30 each time.

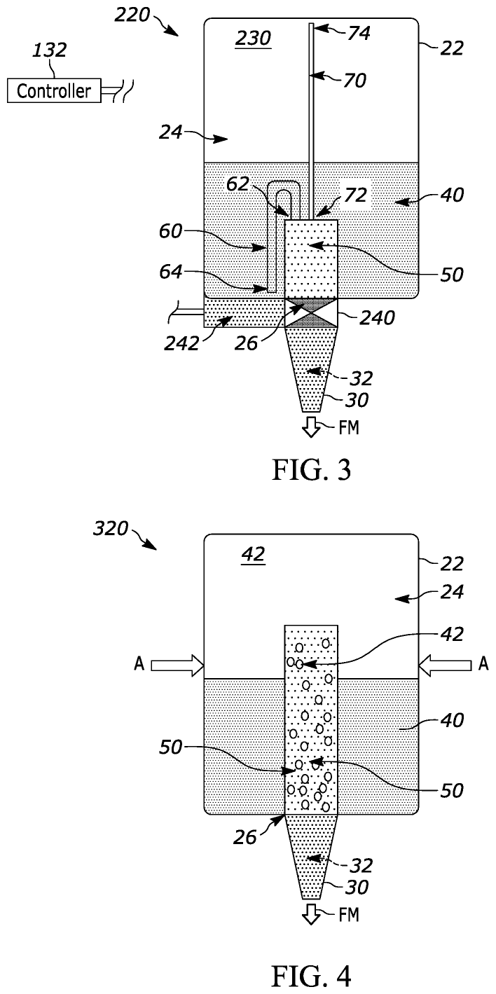

Another example device 220 is shown in FIG. 3. In FIG. 3, features that are the same or substantially the same as those shown in FIG. 1 are given the same reference number. In FIG. 3, the medication 40 is foamed by a pressurized gas 230 stored in the chamber 24. The pressurized gas 230 can be, for example, carbon dioxide or the like. In other words, the pressurized gas 230 replaces the volume of air 42 utilized in the previous embodiments.

A valve 240 is positioned between the foaming chamber 50 and the nozzle 30 to control fluid flow therebetween. To this end, the valve 240 has an initially closed condition preventing fluid flow from the foaming chamber 50/opening 26 in the housing 22 to the passage 32 of the nozzle 30. The valve 240 is connected to the controller 132 and an actuator 242, e.g., a mechanical or electrical actuator, for selectively placing the valve in an open condition allowing fluid to flow from the foaming chamber 50 to the nozzle 30.

In operation, the pressurized gas 230 is initially held within the chamber 24 by the closed valve 240. When the actuator 242 is operated by the controller 132 (or manually by the user) to open the valve 240, the pressurized gas 230 flows through the openings 76 in the second tube 70 and into the foaming chamber 50. At the same time, the pressurized gas 230 also acts on the medication 40 by forcing it through the first tube 60 and into the foaming chamber 50. The pressurized gas 230 combines with the incoming medication 40 and exits the foaming chamber 50, passes through the open valve 240, and exits the nozzle 30 as foamed medication FM. The controller 132 can be configured to open the valve 240 for a predetermined amount of time, e.g., about 1 sec, to deliver a predefined volume of the foamed medication FM to the eye.

Another example device 320 is shown in FIG. 4. In FIG. 4, features that are the same or substantially the same as those shown in FIG. 1 are given the same reference number. In FIG. 4, the medication 40 is foamed by the air 42 residing in the chamber 24 above the medication. The air 42 is pressurized by squeezing the housing 22 in the manner A. In this example, the foaming chamber 50 extends through the medication 40 and into the air 42. In other words, a portion of the foaming chamber 50 is exposed to the air 42.

That said, squeezing the housing 22 in the manner A forces air 42 into the exposed portion of the foaming chamber 50 while simultaneously forcing medication 40 into the submerged portion of the foaming chamber. The foaming chamber 50 in this example is specifically tailored to mix the air 42 and medication 50 in an especially controlled manner.

To this end, the foaming chamber 50 may be constructed of a porous foam or mesh lattice which provides a resistance to liquid such that it tends toward a low liquid saturation with air moving more easily through the lattice than liquid. Such an optimum in resistance to fluid flow may be created, for example, by manipulating the surface energy of a polymer material. Alternatively, a mechanical film with small pores wrapped around a low density foam core may achieve the same goal.

In either case, when the housing 22 is squeezed in the manner A the air 42 is aspirated through the lattice in the foaming chamber 50 and neutralizes the pressure difference in the chamber 24, thereby obviating the need for a one way valve. When the device 320 is at rest, the liquid medication 40 enters the lattice material of the foaming chamber 50 until it reaches an equilibrium state of saturation. When the chamber 24 is thereafter pressurized, the air 42 is preferentially moved through the lattice of the foaming chamber 50 from the top of the device 320 towards the pressure relief at the nozzle 30, and mixes with a small amount of the fluid medication 40 on the way. As a result of this configuration, the separate drug pickup tube 60 and air pickup tube 70 of the prior embodiments can be omitted.

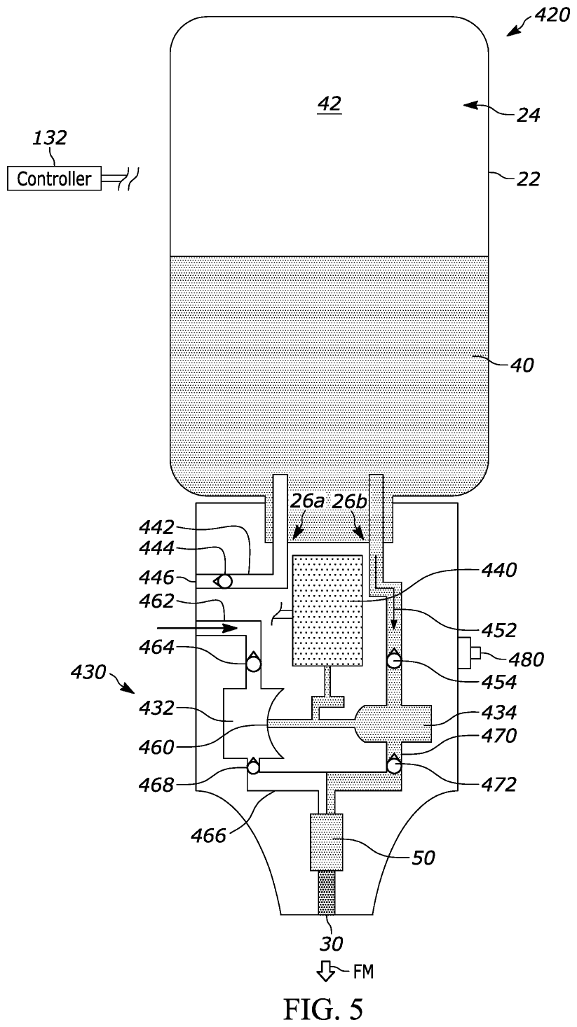

Another example device 420 is shown in FIG. 5. In FIG. 5, features that are the same or substantially the same as those shown in FIG. 1 are given the same reference number. In this example, the medication 40 is pre-mixed in the housing 22 with one or more surfactants. The surfactant can be a nonionic surfactant such as Polysorbate 80, Cremophor EL and/or sorbitan monooleate (Span 80). Specific surfactants or combinations of surfactants can facilitate formation and maintenance of foam as the medication 40 passes through the device 420 while still maintaining the desired therapeutic functions of the medication. In other words, the surfactant helps to promote/enhance foaming of the medication as opposed to suppressing foaming.

The device 420 in FIG. 5 uses an adaptor 430 to mix the medication/surfactant mixture 40 with outside air in order to dispense foamed medication FM. To this end, the adaptor 430 includes an air chamber 432 and a liquid chamber 434. A pump 440 is also provided in the adaptor 430 and connected to the controller 132 for controlling fluid flow to each chamber 432, 434. The pump 440 can be a variable displacement pump. One of the chambers—in this example the air chamber 432—includes a diaphragm (not shown).

A conduit 442 extends through the adaptor 430 from a first opening 26a in the housing 22 to an inlet 446 in the adaptor. A one-way valve 444 is provided in the conduit 442. An inlet conduit 452 extends from a second opening 26b to the liquid chamber 434. A one-way valve 454 is provided in the inlet conduit 452. An outlet conduit 470 extends from the liquid chamber 434 to the foaming chamber 50. A one-way valve 472 is provided in the outlet conduit 470.

An inlet conduit 462 extends from an inlet exposed to outside air to the air chamber 432. A one-way valve 464 is provided in the inlet conduit 462. An outlet conduit 466 extends from the air chamber 432 to the foaming chamber 50. A one-way valve 468 is provided in the outlet conduit 466. The nozzle 30 extends downstream from the foaming chamber 50 and expels foamed medication FM towards the eye. An actuation button 480 can be operated by the user to selectively operate the pump 440 (via the controller 132) and thereby dispense foamed medication FM.

In operation, actuating the pump 440 draws air into the air chamber 432 via the inlet conduit 462. At the same time, liquid medication/surfactant mixture 40 is drawn into the liquid chamber 434 via the inlet conduit 452. The air 42 and medication/surfactant mixture 40 pass separately through the respective outlet conduits 466, 470 and are combined within the foaming chamber 50. When the one of the chambers of the pump 440 has a variable displacement, the liquid-to-air ratio can be varied to accommodate a specific drug formation. In other words, the pump 440 displacement can be adjusted depending on the formulation of the medication/surfactant mixture 40 in order to provide the appropriate liquid-to-air ratio for foaming that particular medication.

When the mixture of air 42 and medication/surfactant mixture 40 passes through the foaming chamber 50, the fine mesh medium reduces the size of the bubbles in the mixture. That said, a desired volume of the mixture exits the nozzle 30 as a foamed medication FM.

The devices shown and described herein are advantageous in that foamed variations of the normally liquid eye medications can be delivered to the user in a repeatable manner while reducing the risk of waste. A foamed medication has greater viscosity than a liquid medication and, thus, the dose delivered has an increased chance of uniformly and fully dispersing over the surface of the eye. A patch of foam stays on the surface of the eye/eyelids for a longer time compared to a standard eye drop for which an excess volume just runs down one's cheek.

The increased viscosity also helps to mitigate the effects that blinking has on undesirably removing medication from the surface of the eye before. More specifically, blinking may not completely remove the foamed drug from the eye. Instead, as bubbles pop and the volume of the foam reduces over time, more drug will get a chance to diffuse into the sclera/cornea. A patch of foam applied to the eye will also have a larger cross-sectional area compared to a liquid, so if targeting is imprecise, the chance of at least a portion of the applied drug getting into the eye increases.

Potentially, the concentration/amount of drug can be reduced due longer time of contact between the formulation and the eye. Moreover, a foamed eye medication may be particularly useful when the eye is prevented from blinking, such as when a speculum is placed in the eye for surgical purposes and the compound should cover and remain on the ocular surface for some amount of time. The duration that the foam persists may be used to enforce an amount of working time for an agent to act on the eye before a next step is taken.

What have been described above are examples of the present invention. It is, of course, not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, modifications and variations that fall within the spirit and scope of the appended claims.

What is claimed is:

1. A device for delivering foamed eye medication to an eye, comprising:
a housing defining an interior space for receiving liquid eye medication and air;
an nozzle having a passage in fluid communication with the interior space; and
a foaming chamber positioned within the interior space and in fluid communication with the nozzle, wherein the air and liquid eye medication are mixed in the foaming chamber and exit the nozzle as foamed medication in response to pressurizing the interior space.

2. The device recited in claim 1, further comprising an air intake tube extending from the foaming chamber and into the air.

3. The device recited in claim 2, wherein the air is urged through the air intake tube and into the foaming chamber while the medication is urged into the foaming chamber in response to pressurizing the interior space.

4. The device recited in claim 3, wherein the interior space is pressurized in response to squeezing the housing.

5. The device recited in claim 3, further comprising a pump for pressurizing the interior space.

6. The device recited in claim 1, further comprising a drug intake tube extending from a first end positioned within the foaming chamber to a second end positioned within the medication.

7. The device recited in claim 1, wherein the foaming chamber comprises at least one of a mesh lattice and a sponge material.

8. The device recited in claim 1, further comprising a one-way valve for allowing the interior space to refill with air.

9. A device for delivering foamed eye medication to an eye, comprising:
a housing defining an interior space for receiving liquid eye medication and pressurized gas;
an nozzle having a passage in fluid communication with the interior space;
a foaming chamber positioned within the interior space and in fluid communication with the nozzle; and
a valve positioned between the foaming chamber and the nozzle, wherein the pressurized gas and the medication mixes within the foaming chamber in response to actuation of the valve and exits the nozzle as foamed medication.

10. The device recited in claim 9, wherein the pressurized gas comprises carbon dioxide.

11. A device for delivering foamed eye medication to an eye, comprising:
a housing defining an interior space for receiving a pre-mixed mixture of liquid eye medication and a surfactant for promoting foaming of the medication; and
an nozzle having a passage in fluid communication with the interior space for delivering foamed medication to the eye.

12. The device recited in claim 11, wherein the surfactant comprises a nonionic surfactant.

13. The device recited in claim 11, wherein the surfactant comprises Polysorbate 80.

14. The device recited in claim 11, wherein the surfactant comprises Cremophor EL.

15. The device recited in claim 11, wherein the surfactant comprises sorbitan monooleate (Span 80).

16. A device for delivering foamed eye medication to an eye, comprising:
a housing defining an interior space for receiving liquid eye medication;
an adapter connected to the housing and including a first chamber for receiving air and a second chamber for receiving the medication;
an nozzle having a passage in fluid communication with the interior space;
a foaming chamber positioned within the adapter and in fluid communication with the nozzle; and
a pump for drawing the air into the first chamber while drawing the medication into the second chamber and subsequently delivering both the air and the medication to the foaming chamber such that the air and the medication mix and exit the nozzle as foamed medication.

17. The device recited in claim 16, wherein the pump has a variable displacement chamber such that the ratio of the air to the medication in the mixture is adjustable.
